(19) United States
(12) Patent Application Publication
(10) Pub. No.: US 2024/0185488 A1
(43) Pub. Date: Jun. 6, 2024

(54) METHODS, SYSTEMS, AND MEDIUMS FOR IMAGE RECONSTRUCTION

(71) Applicant: SHANGHAI UNITED IMAGING HEALTHCARE CO., LTD., Shanghai (CN)

(72) Inventors: Jing YAN, Shanghai (CN); Song ZHANG, Shanghai (CN)

(73) Assignee: SHANGHAI UNITED IMAGING HEALTHCARE CO., LTD., Shanghai (CN)

(21) Appl. No.: 18/531,634

(22) Filed: Dec. 6, 2023

(30) Foreign Application Priority Data
Dec. 6, 2022 (CN) 202211578019.5

(51) Int. Cl. G06T 11/00

(52) U.S. Cl. CPC G06T 11/008; G06T 11/005; G06T 11/006; G06T 2210/41; G06T 2211/436

(57) ABSTRACT

Embodiments of the present disclosure provide a method and system for image reconstruction. The method may include obtaining projection data of a subject corresponding to each of at least one projection angle. The method may also include for the projection data corresponding to each of the at least one projection angle, determining a weight corresponding to each of detection angles within a projection range. The method may further include determining a first derivative of Radon transform data based on the weight and the projection data and determining a reconstructed image of the subject based on the first derivative of the Radon transform data.

### CROSS-REFERENCE TO RELATED APPLICATION

This application claims priority of Chinese Patent Application No. 202211578019.5 filed on Dec. 6, 2022, the contents of which are entirely incorporated herein by reference.

### TECHNICAL FIELD

The present disclosure relates to the field of medical imaging, and in particular, to a method and a system for image reconstruction.

### BACKGROUND

Cone-beam CT imaging technique may focus on obtaining projection data of a subject corresponding to each projection angle based on a movable radiation source. In the medical field, the cone-beam CT imaging technique may be used to reconstruct an image of a subject, subsequent diagnostics or therapies (e.g., CT-guided radiation therapy) may be performed based on a reconstructed image.

The Grangeat reconstruction algorithm may be used in image reconstruction for the cone-beam CT imaging technique. The Grangeat reconstruction algorithm may interpolate and fill shaded areas of Radon transform data due to cone beam acquisition in a traditional FDK algorithm, attenuating or removing cone beam artifacts present in the FDK algorithm, thereby producing a better image reconstruction result. However, the Grangeat algorithm is very complex to handle redundant data generated by projection data, thereby increasing the computation amount.

Therefore, how to reduce a computation amount in a process of cone-beam CT image reconstruction while improving the accuracy of a reconstructed image is a technical problem that needs to be solved.

### SUMMARY

One or more embodiments of the present disclosure provide a method for image reconstruction. The method includes: obtaining projection data of a subject corresponding to each of at least one projection angle; for the projection data corresponding to each of the at least one projection angle, determining a weight corresponding to each of detection angles within a projection range; determining the first derivative of Radon transform data based on the weight and the projection data; and determining a reconstructed image of the subject based on the first derivative of the Radon transform data.

In some embodiments, the weight corresponding to each of the detection angles is determined using a Parker weighted algorithm.

In some embodiments, the weight corresponding to each of the detection angles is determined based on a scanning fan angle within the projection range and the projection angle.

In some embodiments, wherein the determining a weight corresponding to a detection angle within a projection range includes: in response to determining that the projection angle is in within first angle range, determining the weight corresponding to the detection angle based on a difference between the scanning fan angle and the detection angle.

In some embodiments, the first angle range is between 0 and twice of a difference between the scanning fan angle and the detection angle.

In some embodiments, wherein determining a weight corresponding to a detection angle within a projection range includes: in response to determining that the projection angle is within a second angle range, determining the weight to be 1.

In some embodiments, the second angle range is between twice of a difference between the scanning fan angle and the detection angle and a difference between π and twice of the detection angle.

In some embodiments, wherein determining a weight corresponding to a detection angle within a projection range includes: in response to determining that the projection angle is within a third angle range, determining the weight corresponding to the detection angle based on a sum of the scanning fan angle and the detection angle.

In some embodiments, the third angle range is between a difference between π and twice of the detection angle and a sum of π and twice of the scanning fan angle.

In some embodiments, wherein determining the first derivative of Radon transform data based on the weight and the projection data includes: determining, based on a first and a second distance by performing a first weighting operation on the projection data, the weighted projection data; determining, based on the weight and weighted projection data, the first derivative of the Radon transform data, the first distance is a distance from a radiation source to an origin of Radon space, and the second distance is a distance from the radiation source to a point in a line integral direction.

In some embodiments, wherein parameters of the projection data include an angle of a projected image, and obtaining projection data of a subject corresponding to each of at least one projection angle includes: determining, based on an azimuth and an angle difference between a meridian plane and a detector plane, the angle of the projected image.

In some embodiments, wherein determining a reconstructed image of the subject based on the first derivative of the Radon transform data includes: determining, based on the first derivative of the Radon transform data, the second derivative of the Radon transform data; determining, based on the second derivative of the Radon transform data Radon transform data according to an inverse Radon transform operation, the reconstructed image of the subject.

In some embodiments, wherein determining the reconstructed image of the subject based on the second derivative of the Radon transform data according to an inverse Radon transform operation includes: determining, by back-projecting the second derivative of Radon transform data on the meridian plane of 0-π or π-2π according to the inverse Radon transform operation, the reconstructed image of the subject.

One or more embodiments of the present disclosure provide a system, comprising: at least one storage device including a set of instructions; and at least one processor in communication with the at least one storage device, wherein when executing the set of instructions, the at least one processor is configured to direct the system to implement the methods including: obtaining projection data of a subject corresponding to each of at least one projection angle; for the projection data corresponding to each of the at least one projection angle, determining a weight corresponding to each of detection angles within a projection range; determining, based on the weight and the projection data, the first derivative of Radon transform data; and determining, based on the first derivative of the Radon transform data, a reconstructed image of the subject.

One or more embodiments of the present disclosure provide a computer-readable storage medium, the storage medium stores computer instructions, when the computer instructions are executed by the processor to implement the methods including obtaining projection data of a subject corresponding to each of at least one projection angle; for the projection data corresponding to each of the at least one projection angle, determining a weight corresponding to each of detection angles within a projection range; determining, based on the weight and the projection data, the first derivative of Radon transform data; and determining, based on the first derivative of the Radon transform data, a reconstructed image of the subject.

One or more embodiments of the present disclosure determine in advance weights of projection data corresponding to each detection angle (e.g., determining the weights by a Parker weighted algorithm) so that the line integral is determined based on a single projected image while calculating the first derivative of Radon transform data, thereby avoiding the lengthy computation of weights ω₁ and ω₂ in the Grangeat algorithm, and greatly reducing an amount of computation in a process of cone-beam CT image reconstruction while ensuring the accuracy of the reconstructed image.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary application scenario of an image reconstruction system according to some embodiments of the present disclosure;

FIG. 2 is a schematic diagram illustrating an exemplary projection angle of a cone-beam CT short scanning process according to some embodiments of the present disclosure;

FIG. 3 is an optical diagram illustrating an exemplary imaging process at a projection angle according to some embodiments of the present disclosure;

FIG. 4A is another optical diagram illustrating an imaging process at a projection angle according to some embodiments of the present disclosure;

FIG. 4B is an optical diagram illustrating an exemplary meridian plane during an imaging process at a projection angle according to some embodiments of the present disclosure;

FIG. 4C is an optical diagram illustrating an exemplary t-detector plane during an imaging process at a projection angle according to some embodiments of the present disclosure;

FIG. 5 is a flowchart illustrating an exemplary process for reconstructing an image according to some embodiments of the present disclosure;

FIG. 6 is a schematic diagram illustrating exemplary modules of the image reconstruction system according to some embodiments of the present disclosure; and

FIG. 7 is a diagram illustrating a comparison of algorithms between a reference document and some embodiments of the present disclosure.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system”, “device” as used herein, “unit” and/or “module” as used herein is a way to distinguish between different components, elements, parts, sections or assemblies at different levels. However, the words may be replaced by other expressions if other words accomplish the same purpose.

As shown in the present disclosure and the claims, unless the context clearly suggests an exception, the words “one,” “a”, “an”, “one kind”, and/or “the” does not refer specifically to the singular, but may also include the plural. Generally, the terms “including” and “comprising” suggest only the inclusion of clearly identified steps and elements, the steps and elements that do not constitute an exclusive list, and the method or apparatus may also include other steps or elements.

Flowcharts are used in the present disclosure to illustrate operations performed by a system in accordance with embodiments of the present disclosure. It should be appreciated that the preceding or following operations are not necessarily performed in an exact sequence. Instead, steps may be processed in reverse order or simultaneously. Also, it is possible to add other operations to these processes or remove a step or steps from the processes.

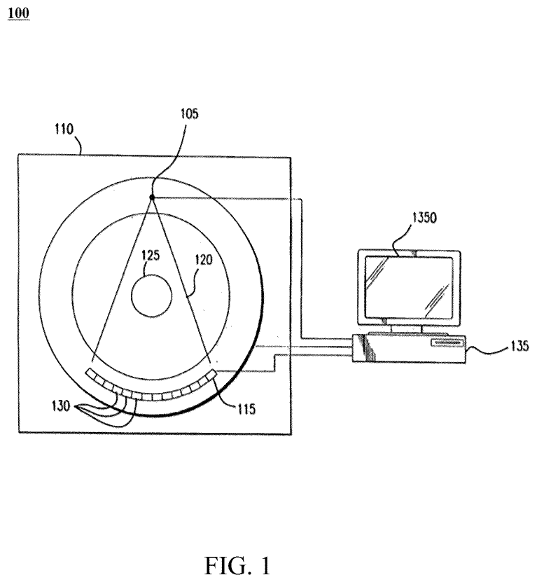

FIG. 1 is a schematic diagram illustrating an image reconstruction system 100 according to some embodiments of the present disclosure.

As shown in FIG. 1, the image reconstruction system 100 may include an imaging device 110 and a processing device 135. During a scanning process, a subject 125 may be placed into a scanning compartment of the imaging device 110, and projection data of the subject 125 corresponding to each of at least one projection angle may be obtained by the imaging device 110, and the projection data corresponding to each of the at least one projection angle may be sent to the processing device 135, and an image reconstruction may be performed on the projection data of the subject 125 corresponding to each of the at least one projection angle to obtain a three-dimensional reconstructed image of the subject 125.

The imaging device 110 may include a cone-beam CT imaging device. For example, the imaging device 110 may include a rack-mounted structural cone-beam CT imaging device, a vertical structural cone-beam CT imaging device, or the like. The imaging device 110 may include a radiation source 105 and a detector 130.

The radiation source 105 may be a radiation device that generates a radiation source (e.g., X-rays). For example, the radiation source 105 may be a hot cathode electron source, an X-ray tube, etc. In some embodiments, the radiation source 105 may move along a predetermined track within the imaging device 110, and emit radiation rays 120 (e.g., X-rays) at one or more predetermined projection angles toward the subject 125 during imaging.

The radiation rays 120 may be imaging rays emitted by the radiation source 105. The radiation rays 120 may be impeded or absorbed by the subject 125 while passing through the subject 125, which causes the radiation rays 120 to change after passing through the subject 125. The intensity of the radiation rays 120 after passing through the subject 125 may be attenuated caused by a density of the subject 125. The greater the density of the subject through which the radiation rays pass, the greater the attenuation of the X-ray intensity may be.

The detector 130 may receive the radiation rays 120 after passing through the subject 125 and convert the received radiation rays 120 into electrical signals. The electrical signals may be converted into projection data of the subject 125 at a certain projection angle. The detector 130 and the radiation source 105 may move relative to each other in the predetermined track, and the radiation rays 120 emitted by the radiation source 105 may propagate to the detector 130. In some embodiments, the detector 130 may include a plurality of detection units 115, and each of the plurality of detection units 115 may correspond to a detection angle for receiving radiation rays corresponding to the detection angle.

In some embodiments, the plurality of detection units 115 may be communicatively coupled to the processing device 135 for sending the projection data of the subject 125 at a detection angle corresponding to the projection angle to the processing device 135.

The processing device 135 may obtain projection data captured by each of at least a portion of the plurality of detection units to determine a projection image of the subject 125 at each projection angle. The radiation source 105 and the detector 130 may be controlled to rotate on the predetermined track to obtain the projection data of the subject 125 at each projection angle. The processing device 135 may reconstruct a three-dimensional reconstructed image of the subject 125 based on obtain based on projection images of the subject 125 at various projection angles.

During a reconstruction process, the processing device 135 may determine a density distribution of the subject 125 at the projection angle (i.e., a Radon change result of the subject 125 at the projection angle) based on the projection data at each projection angle, and then determine a three-dimensional reconstructed image (e.g., a Radon inversion result of the subject 125 at the projection angle) based on the density distribution of the subject 125 at each projection angle (e.g., Radon inverse conversion of the projection data at each projection angle).

In some embodiments, the image reconstruction system 100 may further include a display device 1350. The display device 1350 may present the three-dimensional reconstructed image of the subject 125.

In some embodiments, the image reconstruction system may be used to perform a short scan of the subject 125. The short scan may refer to performing a 0˜(π+2βₘ) scan of the subject 125 in order to save a scanning time instead of performing a 360-degree scan of the subject 125. βₘ is half of a scanning fan angle within a projection range. The projection range refers to a range covered by radiation rays emitted by the radiation source.

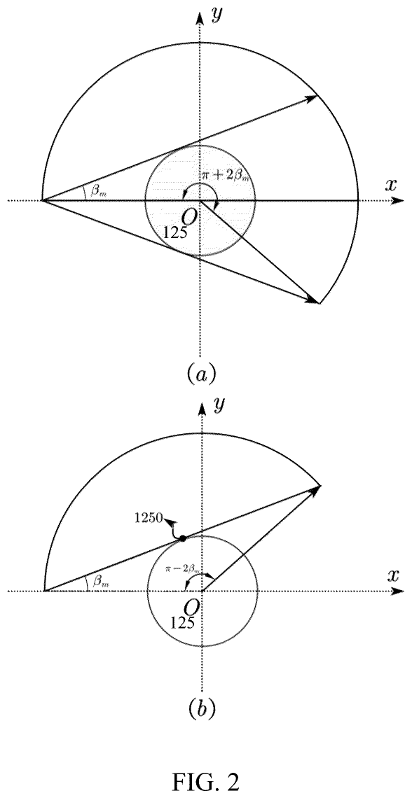

FIG. 2 is a schematic diagram illustrating an exemplary projection angle of a cone-beam CT short scanning process according to some embodiments of the present disclosure.

As shown in FIG. 2(a), after a 0˜(π+2βₘ) scan is performed, each point within the subject 125 is scanned for at least 180 degrees, i.e., an angle between a line connecting any point within the subject 125 and a location of a radiation source when the scan begins and a line connecting the point and a location of the radiation source when the scan ends is greater than or equal to π. Considering that a Radon projection is an integral of an area along a projection direction and Radon projections are numerically the same in opposite projections, i.e., there is no need to perform a scan for a remaining angle, and a reconstructed image of the subject 125 may be reconstructed based on projection data of the 0˜(π+2βₘ) scan.

However, during a short scanning process, there is a plurality of times of sampling, i.e., some portions of the subject 125 are scanned repeatedly. Such repeated scans may generate artifacts.

Taking a tangent point 1250 as an example, as shown in FIG. 2(b), when the radiation source completes a 0˜(π−2βₘ) scan, a x-scan (i.e., a line connecting a track of the radiation source with the tangent point 1250 has equaled π) has been performed on the tangent point 1250 of the subject 125, and when a subsequent (π−2βₘ)˜(π+2βₘ) scan is performed, the tangent point 1250 is repeatedly scanned at a plurality of angles. If an image reproduction is performed directly based on the projection data of the 0˜(π+2βₘ) scan, projection data of the tangent point 1250 at some angles is repeatedly sampled, causing the generation of redundant data and a miscalculation of an intensity of the tangent point 1250 along a redundant direction, resulting in artifacts around the tangent point 1250. Similarly, other points within the subject may be sampled repeatedly, resulting in redundant data.

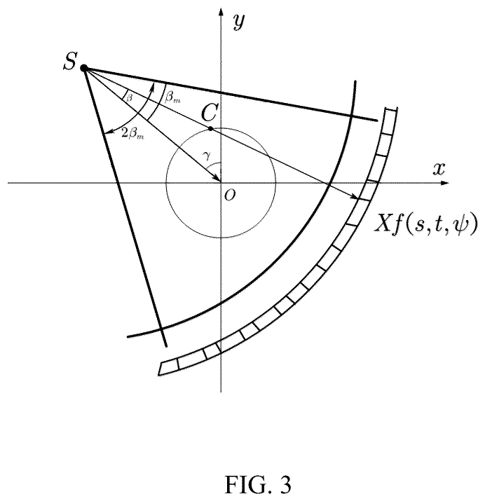

FIG. 3 is an optical diagram illustrating an exemplary imaging process at a projection angle according to some embodiments of the present disclosure.

As shown in FIG. 3, a radiation source may be denoted as S, an origin of a space may be denoted as O, and any point of a subject may be denoted as C. An angle between \vec{OS} and a positive direction of y-axis is a projection angle γ, an angle between \vec{SO} and \vec{SC} is a detection angle β, and an angle βm between \vec{SO} and a line connecting S to an edge of a detector is half of a scanning fan angle within a projection range. The origin of the space may be a center point of the subject. For example, the gravity center of the subject may be used as the origin of the space.

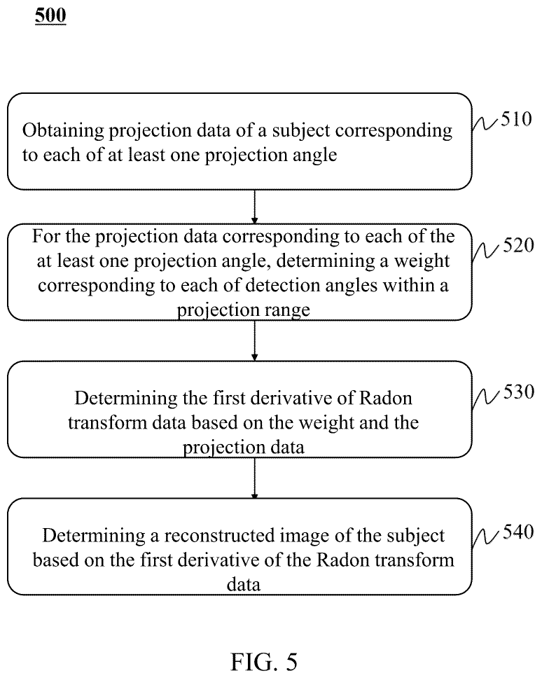

More descriptions for the projection angle, the detection angle, and projection data may be found elsewhere in the present disclosure. See, FIG. 5 and the descriptions thereof.

FIG. 5 is a flowchart illustrating an exemplary process for reconstructing an image according to some embodiments of the present disclosure. In some embodiments, process 500 may be performed by an image reconstruction system 600 or 100. For example, process 500 may be performed by the processing device 135 as described in FIG. 1.

In 510, projection data of a subject corresponding to each of at least one projection angle may be obtained. In some embodiments, operation 510 may be performed by an obtaining module 610 or the processing device 135.

In some embodiments, the subject may be placed into a scanning compartment of an imaging device to obtain the projection data of the subject corresponding to each of the at least one projection angle.

In some embodiments, the subject may be a biological subject. For example, the subject may include a particular portion, organ, and/or tissue of a human body. As another example, the subject may include the head, the neck, the chest, the heart, the stomach, a blood vessel, a soft tissue, a tumor, a nodule, etc., or any combination thereof.

In some embodiments, the subject may be determined based on a subsequent medical operation. For example, if the subsequent medical operation is a CT-guided targeted ablation, the subject may be a portion of a limb where a tumor is located. As another example, for a thyroid nodule, the subject may be the neck of the patient.

The projection angle may refer to an angle indicating a location where a radiation source of radiation rays in the imaging device is located. For example, the projection angle may be characterized as an angle between a vector from an origin O of the Radon space to a radiation source S and a positive direction of a y-axis of the Radon space (e.g., an angle between \vec{OS} and the positive direction of the y-axis in FIG. 3 is denoted as a projection angle γ). In some embodiments, the radiation source of radiation rays may rotate along a predetermined track during a scanning process and emit the radiation rays at a specific location. Each location at which the radiation source emits the radiation rays during the scanning process may correspond to one of the at least one projection angle.

In some embodiments, the at least one projection angle may be denoted as a sequence of equally increasing projection angles (also referred to as a projection sequence). The at least one projection angle may have a maximum value of (π+2βₘ) and a minimum of 0. βm may refer to half of a scanning fan angle within a projection range (e.g., an angle βm between \vec{SO} and the line connecting S to an edge of a detector in FIG. 3). As used herein, the scanning fan angle refers to a maximum angle formed by the radiation rays in a single scan that are received by the detector, i.e., the scanning fan angle includes an angle between two lines connecting the radiation source and edges of the detector on both sides (e.g., 2βm in FIG. 3). In some embodiments, an incremental amount of the equally increasing projection angles may be determined based on an actual accuracy requirement. The higher the accuracy requirement is, the smaller the incremental amount may be. For example, the incremental amount of the projection angle may be 5 degrees, and βm may be 30 degrees, and then the at least one projection angle may be characterized as an equidistant series from 0 degrees to 240 degrees with a difference of 5 degrees.

The projection data may be generated by the detector receiving the radiation rays and converting the received radiation rays into electrical signals. The detector may include a plurality of detector units, and different detector units may generate projection data corresponding to different detection angles.

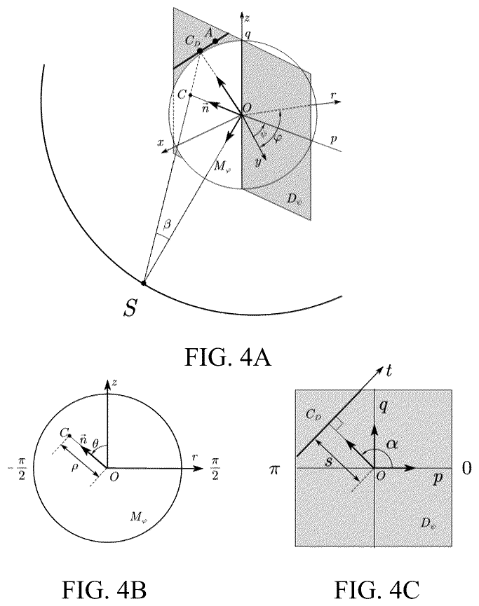

As shown in FIG. 3, after the radiation rays emitted by the radiation source pass through a point C on the subject, radiation rays in a direction of \vec{SC} are received by one of the detector units for generating projection data based on the received radiation rays, and the projection data may be written as Xf(s,t,ψ), where f(s, t, ψ) refers to a subject density function, i.e., 3D data that needs to be reconstructed. As shown in in FIG. 4A, the projection data of the subject in the direction of \vec{SC} received by the detector may be denoted as Xf(s,t,ψ), representing a line integral of f (s,t,ψ) in a detector plane Dψ along a straight line t. As shown in FIG. 4C, t represents a straight line perpendicular to OC_{D} on the detector plane Dψ with an angle ψ to the y-axis, i.e., a line integral direction, C_{D} represents an intersection of \vec{SC} and Dψ, and s represents a distance from a point O to a point C_{D}, i.e., a length of OC_{D}. Axes of the detector plane Dψ are a p-axis and a q-axis, and the angle between Dψ and the y-axis is an angle between the p-axis and the y-axis i.e., ψ. Coordinate parameters of the detector plane Dψ of a feature point C may be denoted as (s,α,ψ), and a denotes an angle between the p-axis and OC_{D}.

In some embodiments, in obtaining the projection data corresponding to each of the at least one projection angle, a location of the radiation source of the radiation rays may be adjusted sequentially according to each of the at least one projection angle, thereby determining the projection data corresponding to each of the at least one projection angle. For example, for the projection angle sequence being an equidistant series of numbers ranging from 0 degrees to 240 degrees with a difference of 5 degrees, the radiation source of the radiation rays may be placed at a location corresponding to the projection angle of 0 degrees first to obtain projection data corresponding to the projection angle of 0 degrees. After completing the scanning, the radiation source of the radiation rays is placed at the location corresponding to the projection angle of 0 degrees, the location of the radiation source of the radiation rays may be adjusted such that the projection angle is changed to the next one in the projection angle sequence to obtain the projection data corresponding to the next projection angle in the projection angle sequence (e.g., 5 degrees). Accordingly, the projection data corresponding to each projection angle may be obtained.

In 520, for the projection data corresponding to each of the at least one projection angle, a weight corresponding to each of the detection angles within the projection range may be obtained. In some embodiments, operation 520 may be performed by a weight determination module 620 or the processing device 135.

The detection angle may refer to an orientation of any point of the subject in the imaging device with respect to the radiation source. For example, in a Cartesian coordinate system, the detection angle may be characterized as an angle between a line connecting the radiation source and the origin of a space and a line connecting the radiation source and any point on the subject (e.g., an angle between \vec{SO} and \vec{SC} in FIG. 3. is denoted as the detection angle β).

In some embodiments, the detection angles may be discretized based on each detection unit of the detector. For example, if each individual detection unit may detect projection data within a detection angle of a certain range, the detection angles may be discretized based on the range of the detection angle of each detection unit. For example, if each detection unit may detect the projection data corresponding to a detection angle of 2 degrees, the detection angles may be characterized as equally incremental data with an incremental amount of 2 degrees, a minimum value of −βm, and a maximum value of βm. Each of the detection angles may correspond to one of the plurality of detection units.

The weight may refer to a weighted weight of the projection data corresponding to each detection angle in a subsequent Radon inverse conversion process. In some embodiments, a sum of weights of opposite detection angles may be 1. The opposite detection angles may be referred to as two detection angles when \vec{S_{β1}A}=−\vec{S_{β2}A}. For example, in FIG. 2(b), the detection angle(β=βₘ) corresponding to the tangent point 1250 when γ=0 and the detection angle (β=βₘ) corresponding to the tangent point 1250 when γ=π−2βm are opposite detection angles, and a sum of weights of the two detection angles may be 1.

When there is no opposite detection angle for a detection angle, a weight of the detection angle is 1. For example, when γ∈(4βm, π−2βₘ), there is no opposite detection angle for the detection angle corresponding to the tangent point 1250, and a weight of the detection angle corresponding to the tangent point 1250 is 1.

In some embodiments, the weight may be determined using a Parker weighted algorithm. The Parker weighted algorithm may be the same as a weighting algorithm described by Dennis L. Parker in the paper: D. L. Parker (1982). Optimal short scan convolution reconstruction for fan beam CT; DOI: 10.1118/1.595078.

In some embodiments, the weight corresponding to each of the detection angles may be determined based on a scanning fan angle within the projection range and the projection angle. Based on an optical structure shown in FIG. 3, the weight corresponding to each of the detection angles may be determined based on a following Equation (1):

$${w}_{γ} ( β ) = \{ \begin{matrix}{sin}^{2} ( \frac{π}{4} \cdot \frac{γ}{{β}_{m} - β} ) , & 0 \leq γ \leq 2 {β}_{m} - 2 β \\ 1 , & 2 {β}_{m} - 2 β \leq γ \leq π - 2 β \\ {sin}^{2} ( \frac{π}{4} \cdot \frac{π + 2 {β}_{m} - γ}{{β}_{m} + β} ) , & π - 2 β \leq γ \leq π + 2 {β}_{m}\end{matrix} , \qquad ( 1 )$$

where w_{γ}(β) denotes a weight function corresponding to each of the detection angles, γ denotes a projection angle corresponding to projection data, βm is half of the scanning fan angle within the projection range, and β denotes the detection angle.

In some embodiments, in response to determining that the projection angle is in a first angle range, the weight corresponding to the detection angle may be determined based on a difference between the scanning fan angle and the detection angle. In some embodiments, the first angle range may be between 0 and twice of the difference between the scanning fan angle and the detection angle. For example, the weight corresponding to the detection angle may be determined according to the following Equation (2):

$$When 0 \leq γ \leq 2 {β}_{m} - 2 β , {w}_{γ} ( β ) = {sin}^{2} ( \frac{π}{4} \cdot \frac{γ}{{β}_{m} - β} ) . \qquad ( 2 )$$

In some embodiments, in response to determining that the projection angle is in a second angle range, the weight may be determined to be 1. In some embodiments, the second angle range may be between twice of the difference between the scanning fan angle and the detection angle and a difference between π and twice of the detection angle. For example, the weight corresponding to the detection angle may be determined according to the following Equation (3):

When 2βₘ−2β≤γ≤π−2β, w_{γ}(β)=1. (3)

In some embodiments, in response to determining that the projection angle is in a third angle range, the weight corresponding to the detection angle may be determined based on a sum of the scanning fan angle and the detection angle. In some embodiments, the third angle range may be between the difference between π and twice of the detection angle and a sum of π and twice of the scanning fan angle. For example, the weight corresponding to the detection angle may be determined according to the following Equation (4):

$$When π - 2 β \leq γ \leq π + 2 {β}_{m} , {w}_{γ} ( β ) = {sin}^{2} ( \frac{π}{4} \cdot \frac{π + 2 {β}_{m} - γ}{{β}_{m} + β} ) . \qquad ( 4 )$$

In some embodiments, the weight may also be determined based on other short scan weighted algorithms. For example, the weight may be determined based on other short scan weighted algorithms that satisfy a continuity condition. As another example, the weight may be determined by modifying function values within a range of 0≤γ≤2βₘ−2β and π−2β≤γ≤π+2βₘ in the Parker weighted algorithm. Modifications such as these are within the scope of protection of the present disclosure.

It should be noted that during the imaging process of cone-beam CT, the X-ray emitted by the radiation source is in the form of a cone beam in three-dimensional space. A spatial relationship described in the preceding section is only a migration of a three-dimensional relationship in x-y coordinates, and the foregoing spatial relationship may also be applied to y-z coordinates, x-z coordinates, and x-y-z coordinates, depending on an actual situation. The weight of each detection angle also holds in a 3D space.

In some embodiments, after obtaining the projection data and the weight, the projection data may be processed based on subsequent operations to determine a three-dimensional reconstructed image.

In some embodiments, the subsequent operations may include two stages. The first stage may be used to obtain the first derivative of Radon transform data by processing the projection data based on the weight to characterize the subject in a Radon space. The second stage may be used to perform a Radon inverse conversion on the first derivative of Radon transform data to obtain a three-dimensional reconstructed image. More information about the first stage may be found in operation 530. More information about the second stage may be found in operation 540.

In 530, determining the first derivative of Radon transform data based on the weight and projection data. In some embodiments, operation 530 may be performed by a first derivative determination module 630 or the processing device 135.

The first derivative of Radon transform data may be a result in the first stage. In some embodiments, determining the first derivative of Radon transform data based on the weight and the projection data may include determining weighted projection data by performing a first weighting operation on the projection data based on a first distance and a second distance; and performing a second weighting operation on the weighted projection data based on the weights to determine the first derivative of Radon transform data.

The first distance refers to a distance from the radiation source to an origin of Radon space, e.g., SO as shown in FIG. 4A. The second distance refers to a length of a projected ray from the radiation source to a point in a line integral direction, e.g., SA (as shown in FIG. 4A, where A is any point on a straight line t). The weighted projection data may be determined according to the following Equation (5):

$${X}_{w} f ( s ( ρ \vec{n} ) , t , Ψ ( ρ \vec{n} ) ) = \frac{SO}{SA} \cdot Xf ( s ( ρ \vec{n} ) , t , Ψ ( ρ \vec{n} ) ) , \qquad ( 5 )$$

where Xf(s(ρ\vec{n}), t, Ψ(ρ\vec{n})) represents the projected data, and X_{w}f(s(ρ\vec{n}), t, Ψ(ρ\vec{n})) represents the weighted projected data after the first weighting operation.

In some embodiments, parameters of the projection data may include an angle Ψ(ρ\vec{n}) of a projected image, and determining the angle of the projected image may include determining the angle of the projected image based on an azimuth and an angle difference between the detector plane and a meridian plane. The angle of the projected image may be determined according to the following Equation (6) and Equation (7):

$$Ψ ( ρ \vec{n} ) = Ψ ( ρ , θ , φ ) = φ + {sin}^{- 1} [ \frac{ρ}{SO sin θ} ] , \qquad ( 6 )$$

$$or$$

$$Ψ ( ρ \vec{n} ) = Ψ ( ρ , θ , φ ) = φ + π - {sin}^{- 1} [ \frac{ρ}{SO sin θ} ] , \qquad ( 7 )$$

where φ represents the azimuth and

$${sin}^{- 1} [ \frac{ρ}{SO sin θ} ]$$

represents the angle difference between the meridian plane and the detector plane. As shown in FIG. 4B, spherical coordinate parameters of a feature point C in the Radon space may be denoted as (ρ,θ,φ), where \vec{n} denotes a unit vector passing through the feature point. Specifically, ρ represents a radial distance from the origin to the feature point, θ represents a polar angle between a line from the origin to the feature point and a positive z-axis, and φ represents an azimuth between a projection line of the line from the origin to the feature point on a xy-plane and a positive y-axis.

It should be noted that the angle Ψ(ρ\vec{n}) of the projected image may be determined according to one of Equations (6) and (7).

In some embodiments, weighted projection data X_{w}f(s(ρ\vec{n}), t, Ψ(ρ\vec{n})) may be rearranged for calculation based on weights. The first derivative of Radon transform data may be determined based on a following Equation (8):

$$\frac{\partial }{\partial ρ} Rf ( ρ \vec{n} ) = {R}^{′} f ( ρ \vec{n} ) = \frac{1}{{cos}^{2} β} \cdot \frac{\partial }{\partial s} {\int }_{- ∞}^{+ ∞} {P}_{w} \cdot {X}_{w} f ( s ( ρ \vec{n} ) , t , Ψ ( ρ \vec{n} ) ) dt , \qquad ( 8 )$$

where Rf(ρ\vec{n}) represents the Radon transform data, and ∂/∂ρRf(ρ\vec{n})and R′f(ρ\vec{n}) represent the first derivative of the Radon transform data, and P_{w} represents the weights. If

$$Ψ ( ρ \vec{n} ) ∉ [ 0 , π + 2 {γ}_{m} ] , \frac{\partial }{\partial ρ} Rf ( ρ \vec{n} ) = 0.$$

In some embodiments, the first derivative of the Radon transform data described above may be derived based on the Grangeat algorithm in conjunction with the weights. The first derivative of Radon transform data may be determined according to following operations.

S1, the weight of the projection data may be obtained.

S2, a first weighting operation may be performed on the projection data based on SO/SA.

S3, the projection data may be derived in row and column directions to obtain the derivatives of the projection data in the row and column directions, and the sum of the derivatives in the row and column directions may be designated as a derivative of the projection data. The projection data may be derived in row and column directions According to following Equation (7):

$$\frac{\partial }{\partial s} {X}_{w} f [ s ( ρ \vec{n} ) , t , Ψ ( ρ \vec{n} ) ] = cos [ α ( ρ \vec{n} ) ] \frac{\partial }{\partial p} {X}_{w} f [ s ( ρ \vec{n} ) , t , Ψ ( ρ \vec{n} ) ] + sin [ α ( ρ \vec{n} ) ] \frac{\partial }{\partial q} {X}_{w} f [ s ( ρ \vec{n} ) , t , Ψ ( ρ \vec{n} ) ] , \qquad ( 9 )$$

where p and q represent x-axis and y-axis of Cartesian coordinate axes, respectively, and s and a define a polar coordinate system on the detector plane. More descriptions for p, q, s, and a may be found in FIG. 4C and FIG. 1(b) of a reference titled “Grangeat-type half-scan algorithm for cone-beam CT”.

S4, parameters related to the line integral for determining g s, α, and Ψ may be obtained. More descriptions for determining Y may be found in operation 540.

S5, the line integral of the projection data may be determined to obtain the first derivative of the Radon transform data.

In some embodiments, given that the radiation source does not move in the z-axis, a shaded area may exist (i.e., areas where the detector does not pick up data) in the z-axis when the con-beam CT scan is performed. As a result, after determining the first derivative of the Radon transform data, values of the first derivative of the Radon transform data in the shaded area may be obtained based on interpolation to obtain the first derivative of Radon transform data that includes values of the shaded area. An interpolation algorithm may include a nearest neighbor point interpolation manner and a linear interpolation manner.

In 540, a reconstructed image of the subject may be determined based on the first derivative of the Radon transform data. In some embodiments, operation 540 may be performed by an image reconstruction module 640 or the processing device 135.

A three-dimensional reconstructed image may refer to a modeling of the subject in the 3D space. That is, the projection data in the Radon space may be transformed into graphic data in the 3D space. For example, when the subject is a part of a human body, a three-dimensional reconstructed image after reconstruction may include a three-dimensional image representing the part and internal tissues thereof. In some embodiments, after determining the three-dimensional reconstructed image, actual meanings of regions may be labeled in the three-dimensional reconstructed image based on densities of various parts of the subject in the reconstructed image and the subject.

In some embodiments, a second derivative of the Radon transform data may be determined based on the first derivative of the Radon transform data; the three-dimensional reconstructed image of the subject may be determined based on the second derivative of the Radon transform data according to a Radon inverse transform operation. For example, the first derivative of the Radon transform data may be filtered first to obtain the second derivative of the Radon transform data. The filtering manner may include Gaussian filtering, etc. Based on the second derivative of the Radon transform data, the three-dimensional reconstructed image of the subject may be determined according to the Radon inverse transform operation. The Radon inverse transform operation may be performed according to the following Equation (10):

$$f ( ρ \vec{n} ) = \frac{- 1}{8 {π}^{2}} {\int }_{- ∞}^{+ ∞} {\int }_{- ∞}^{+ ∞} \frac{{\partial }^{2}}{\partial {ρ}^{2}} Rf [ ( ρ \vec{n} ) \vec{n} ] \vert sin θ \vert d φ d θ , \qquad ( 10 )$$

where after determining the first derivatives of the Radon transform data on all meridian planes, the first derivative of the Radon transform data on a meridian plane of 0-π and the first derivative of the Radon transform data on a meridian plane of π-2π may be added. In a second stage of the algorithm, the reconstructed image of the subject may be determined by only back projecting the second derivative of Radon transform data on the meridian plane of 0-π or π-2π according to an inverse Radon transform operation. The inverse Radon transform operation may be performed according to the following Equation (11) and Equation (12):

$$f ( ρ \vec{n} ) = \frac{- 1}{8 {π}^{2}} \cdot {\int }_{θ = \frac{- π}{2}}^{θ = \frac{π}{2}} {\int }_{φ = 0}^{φ = π} \frac{{\partial }^{2}}{\partial {ρ}^{2}} Rf [ ( ρ \vec{n} ) \vec{n} ] \vert sin θ \vert d φ d θ , \qquad ( 11 )$$

$$or$$

$$f ( ρ \vec{n} ) = \frac{- 1}{8 {π}^{2}} \cdot {\int }_{θ = \frac{- π}{2}}^{θ = \frac{π}{2}} {\int }_{φ = π}^{φ = 2 π} \frac{{\partial }^{2}}{\partial {ρ}^{2}} Rf [ ( ρ \vec{n} ) \vec{n} ] \vert sin θ \vert d φ d θ , \qquad ( 12 )$$

where R(φ, ρ, θ)=R₁(φ, ρ, θ)+R₂(φ+π, ρ, −θ).

In the method for cone-beam CT image reconstruction provided on the basis of some embodiments of the present disclosure, the weight of each detection angle is determined before determining the first derivative of the Radon transform data, so that in determining the first derivative of the Radon transform data, a line integral of an angle of a single projected image may be processed rather than processing line integrals of angles of a plurality of projected images, which significantly reduces the algorithm's complexity.

In some embodiments, the reconstructed image may be used as a scout image. The scout image may be used to determine one or more scanning parameters and/or one or more treatment parameters of the subject. For example, the reconstructed image may be used to determine a scanning range, a scanning direction, etc., of the subject. As another example, the reconstructed image may be used to determine a tube voltage, a tube current, etc., for the scanning of the subject. As another example, the reconstructed image may be used to guide the positioning of the subject in a treatment process. As another example, the reconstructed image may be used to determine at least a portion of a radiotherapy plan.

In some embodiments, based on the reconstructed image, subsequent diagnosis and/or treatment may be performed on the subject. For example, one or more regions of interest (ROIs) may be identified from the reconstructed image and a type of the one or more ROIs may be determined based on characteristics of the ROIs identified from the reconstructed image. In some embodiments, a neural network model may be used to segment the ROIs from the reconstructed image and/or determine a type of the one or more ROIs.

In some embodiments, the radiotherapy plan may be performed on at least one of the ROIs.

As a further example, the reconstructed image may be used for the diagnosis of root longitudinal rows, the localization of foreign bodies in the root canal, etc. As still another example, in a radiotherapy of pelvic tumors, the reconstructed image may be used to accurately distinguish pelvic soft tissue structures and to control posing errors during the radiotherapy.

In some embodiments, the reconstructed image may be sent to a terminal device for display. For example, the terminal device may include a user interface and a display device. The user interface may receive the reconstructed image from the processing device and cause the display device to display the reconstructed image.

In some embodiments, a three-dimensional model of the subject may be constructed based on the reconstructed image according to a 3D reconstruction technique. The one or more ROIs may be highlighted on the 3D model of the subject and displayed on the display device. In some embodiments, the user interface may receive an input of an operator and adjust the range of an ROI and/or the 3D model of the subject based on the input of the operator.

In some embodiments, one or more reconstruction parameters (e.g., a projection angle, a detection angle, the weight, etc.) as described in FIG. 5 may be adjusted according to an input of the user inputted through the user interface.

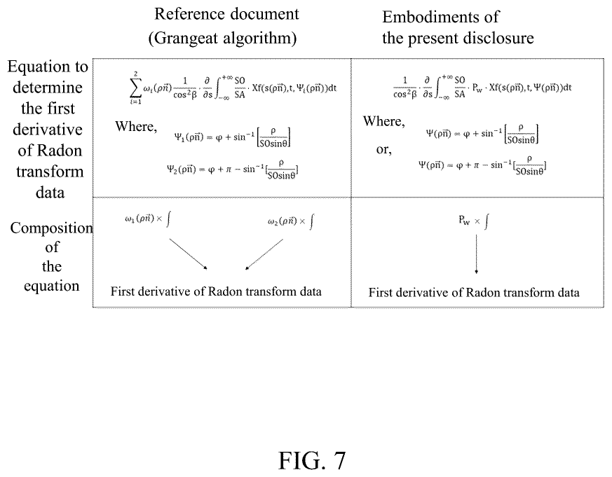

In order to further illustrate the beneficial effects of embodiments of the present disclosure, FIG. 7 provides an algorithmic comparison of the Grangeat algorithm with some embodiments of the present disclosure.

As shown in FIG. 7, the first column of the table shows a formula to determine the first derivative of Radon transform data in the Grangeat algorithm and equations used in the Grangeat algorithm. In the Grangeat algorithm, the first derivative of the Radon transform data may be determined by a following Equation (13):

$$\frac{\partial }{\partial ρ} Rf ( ρ \vec{n} ) = {\sum }_{i = 1}^{2} {ω}_{i} ( ρ \vec{n} ) \frac{1}{{cos}^{2} β} \cdot \frac{\partial }{\partial s} {\int }_{- ∞}^{+ ∞} \frac{SO}{SA} Xf ( s ( ρ \vec{n} ) , t , {Ψ}_{i} ( ρ \vec{n} ) ) dt \qquad ( 13 )$$

$${Ψ}_{1} ( ρ , θ , φ ) = φ + {sin}^{- 1} [ \frac{ρ}{SO sin θ} ]$$

$${Ψ}_{2} ( ρ , θ , φ ) = φ + π - {sin}^{- 1} [ \frac{ρ}{SO sin θ} ] ,$$

Different from the first derivative of Radon transform data provided in the present disclosure, the above equation is derived without performing the above operation S1, and angles of two projected images are determined in operation S4.

As can be seen from the above formula based on the prior art in calculating the first derivative of the Radon transform data, it is necessary to determine line integrals of the two projected images, i.e., the formula for calculating the first derivative of the Radon transform data is a combination of data of the two projected images and their corresponding weights, and the determination of the weights ω₁ and ω₂ is very complicated in the Grangeat algorithm.

The second column of the table presents equations used in the first derivative of the Radon transform data in embodiments of the present disclosure. In embodiments of the present disclosure, the first derivative of the Radon transform data may be determined by a following Equation (14):

$$\frac{\partial }{\partial ρ} Rf ( ρ \vec{n} ) = {R}^{′} f ( ρ \vec{n} ) = \frac{1}{{cos}^{2} β} \cdot \frac{\partial }{\partial s} {\int }_{- ∞}^{+ ∞} \frac{SO}{SA} \cdot {P}_{w} \cdot {X}_{w} f ( s ( ρ \vec{n} ) , t , Ψ ( ρ \vec{n} ) ) dt \qquad ( 14 )$$

$$where ,$$

$$Ψ ( ρ \vec{n} ) = Ψ ( ρ , θ , φ ) = φ + {sin}^{- 1} [ \frac{ρ}{SO sin θ} ]$$

$$or$$

$$Ψ ( ρ \vec{n} ) = Ψ ( ρ , θ , φ ) = φ + π - {sin}^{- 1} [ \frac{ρ}{SO sin θ} ] ,$$

The embodiment of the present disclosure achieves the effect of determining the weights in advance by executing operation SI described above, i.e., performing a Parker weighting on the projection data in advance, so that only one projected image is required for determining the line integral while determining the first derivative of the Radon transform data, thereby avoiding a lengthy calculation of the weights ω₁ and ω₂ in the Grangeat algorithm and greatly reducing an amount of operations, thereby decreasing the computation burden.

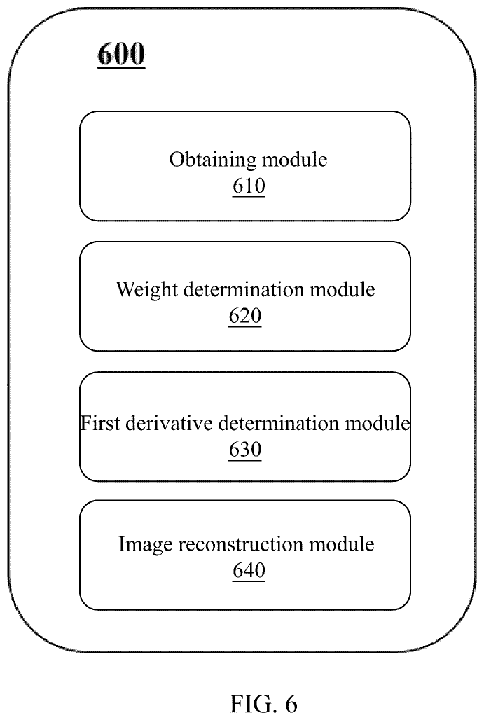

FIG. 6 is a schematic diagram illustrating exemplary modules of an image reconstruction system according to some embodiments of the present disclosure.

As shown in FIG. 6, the image reconstruction system 600 may include an obtaining module 610, a weight determination module 620, a first derivative determination module 630, and an image reconstruction module 640.

The obtaining module 610 may be used to obtain projection data of a subject corresponding to each of at least one projection angle. More information about the projection data may be found in operation 510 and its related description.

The weight determination module 620 may be used to determine a weight corresponding to each of the detection angles within a projection range for the projection data corresponding to each of the at least one projection angle. More information about the weight may be found in operation 520 and its related description.

The first derivative determination module 630 may be used to determine the first derivative of the Radon transform data by determining the line integration based on the weight. In some embodiments, the weight determination module 630 may be further used to determine the weight using a Parker weighted algorithm. More information about the first derivative of the Radon transform data may be found in operation 530 and its related description.

The image reconstruction module 640 may be used to determine a three-dimensional reconstructed image of the subject based on the first derivative of the Radon transform data. More information about the three-dimensional reconstructed image may be found in operation 540 and its related description.

Some embodiments of the present disclosure further provide a device for image reconstruction, comprising at least one processor and at least one storage device. The at least one storage device may be used to store computer instructions. The at least one processor may be used to execute at least a portion of the computer instructions to implement a method for image reconstruction.

Some embodiments of the present disclosure further provide a computer-readable storage medium storing computer instructions, and implement the method for image reconstruction when the computer instructions are executed by the processor.

It is to be noted that the above description of the system 600 for image reconstruction and its modules is provided only for descriptive convenience, and does not limit the present disclosure to the scope of the cited embodiments. It is to be understood that for a person skilled in the art, after understanding the principle of the system, it may be possible to arbitrarily combine the individual modules or form a sub-system to be connected to the other modules without departing from the principle. In some embodiments, the projection data obtaining module 610, the weight determination module 620, the first derivative determination module 630, and the image reconstruction module 640 disclosed in FIG. 6 may be different modules in a single system module, or it may be a single module that realizes functions of two or more of the above-described modules. For example, the individual modules may share a common storage module, and the individual modules may each have a respective storage module. Deformations such as these are within the scope of protection of the present disclosure.

The basic concepts have been described above, and it is apparent to those skilled in the art that the foregoing detailed disclosure is intended as an example only and does not constitute a limitation of the present disclosure. While not expressly stated herein, various modifications, improvements, and amendments may be made to the present disclosure by those skilled in the art. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure, such as “an embodiment”, “one embodiment”, and/or “some embodiment”, which means a feature, structure, or characteristic associated with at least one embodiment of the present disclosure. Accordingly, it should be emphasized and noted that “an embodiment” or “one embodiment” or “an alternative embodiment” in different locations in the present disclosure do not necessarily refer to the same embodiment. In addition, certain features, structures, or characteristics in one or more embodiments of the present disclosure may be suitably combined.

Furthermore, unless expressly stated in the claims, the order of the processing elements and sequences described herein, the use of numerical letters, or the use of other names are not intended to qualify the order of the processes and methods of the present disclosure. While some embodiments of the invention that are currently considered useful are discussed in the foregoing disclosure by way of various examples, it should be appreciated that such details serve only illustrative purposes, and that additional claims are not limited to the disclosed embodiments, rather, the claims are intended to cover all amendments and equivalent combinations that are consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be noted that in order to simplify the presentation of the present disclosure, and thereby aid in the understanding of one or more embodiments of the invention, the foregoing descriptions of embodiments of the present disclosure sometimes group multiple features together in a single embodiment, accompanying drawings, or a description thereof. However, this method of disclosure does not imply that the subjects of the present disclosure require more features than those mentioned in the claims. Rather, claimed subject matter may lie in less than all features of a single foregoing disclosed embodiment.

Some embodiments use numbers describing the number of components, attributes, and it is to be understood that such numbers used in the description of embodiments are modified in some examples by the modifiers “about”, “approximately”, or “substantially”. Unless otherwise noted, the terms “about,” “approximately,” or “roughly” indicates that a ±20% variation in the stated number is allowed. Correspondingly, in some embodiments, the numerical parameters used in the present disclosure and claims are approximations, which can change depending on the desired characteristics of individual embodiments. In some embodiments, the numerical parameters should take into account the specified number of valid digits and employ general place-keeping. While the numerical domains and parameters used to confirm the breadth of their ranges in some embodiments of the present disclosure are approximations, in specific embodiments, such values are set to be as precise as possible within the feasible range.

For each patent, patent application, patent application disclosure, and other material cited in the present disclosure, such as articles, books, specification sheets, publications, documents, and the like, the entire contents of which are hereby incorporated herein by reference. Historical application history documents that are inconsistent with or create a conflict with the contents of the present disclosure are excluded, as well as documents that limit the broadest scope of the claims of the present disclosure (currently or hereafter appended to the present disclosure). It should be noted that to the extent that the descriptions, definitions, and/or use of terms in the materials appended to the present disclosure are inconsistent with or in conflict with the contents of the present disclosure, the descriptions, definitions and/or use of terms in the present disclosure prevail.

Finally, it should be understood that the embodiments described in the present disclosure are only used to illustrate the principles of the embodiments of the present disclosure. Other deformations may also fall within the scope of the present disclosure. As such, alternative configurations of embodiments of the present disclosure may be viewed as consistent with the teachings of the present disclosure as an example, not as a limitation. Correspondingly, the embodiments of the present disclosure are not limited to the embodiments expressly presented and described herein.

What is claimed is:

1. A system, comprising:
at least one storage device including a set of instructions; and
at least one processor in communication with the at least one storage device, wherein when executing the set of instructions, the at least one processor is configured to direct the system to implement a method including:
obtaining projection data of a subject corresponding to each of at least one projection angle;
for the projection data corresponding to each of the at least one projection angle, determining a weight corresponding to each of detection angles within a projection range;
determining, based on the weight and the projection data, a first derivative of Radon transform data; and
determining, based on the first derivative of the Radon transform data, a reconstructed image of the subject.

2. The system of claim 1, wherein the weight corresponding to each of the detection angles is determined using a Parker weighted algorithm.

3. The system of claim 2, wherein the weight corresponding to each of the detection angles is determined based on a scanning fan angle within the projection range and the projection angle.

4. The system of claim 3, wherein the determining a weight corresponding to a detection angle within the projection range includes:
in response to determining that the projection angle is within a first angle range, determining the weight corresponding to the detection angle based on a difference between the scanning fan angle and the detection angle.

5. The system of claim 4, wherein the first angle range is between 0 and twice of a difference between the scanning fan angle and the detection angle.

6. The system of claim 3, wherein the determining a weight corresponding to a detection angle within the projection range includes:
in response to determining that the projection angle is within a second angle range, determining the weight to be 1.

7. The system of claim 6, wherein the second angle range is between twice of a difference between the scanning fan angle and the detection angle and a difference between π and twice of the detection angle.

8. The system of claim 3, wherein the determining a weight corresponding to a detection angle within the projection range includes:
in response to determining that the projection angle is within a third angle range, determining the weight corresponding to the detection angle based on a sum of the scanning fan angle and the detection angle.

9. The system of claim 8, wherein the third angle range is between a difference between π and twice of the detection angle and a sum of π and twice of the scanning fan angle.

10. The system of claim 1, wherein the determining, based on the weight and the projection data, the first derivative of the Radon transform data includes:
determining, based on a first distance and a second distance by performing a first weighting operation on the projection data, weighted projection data;
determining, based on the weight and the weighted projection data, the first derivative of the Radon transform data;
wherein the first distance is a distance from a radiation source to an origin of Radon space, and the second distance is a distance from the radiation source to a point in a line integral direction.

11. The system of claim 1, wherein a parameter of the projection data includes an angle of a projected image, and the obtaining projection data of a subject corresponding to each of at least one projection angle includes:
determining, based on an azimuth and an angle difference between a meridian plane and a detector plane, the angle of the projected image.

12. The system of claim 1, wherein the determining, based on the first derivative of the Radon transform data, a reconstructed image of the subject includes:
determining, based on the first derivative of the Radon transform data, a second derivative of the Radon transform data; and
determining, based on the second derivative of the Radon transform data, the reconstructed image of the subject according to an inverse Radon transform operation.

13. The system of claim 12, wherein the determining, based on the second derivative of the Radon transform data, the reconstructed image of the subject according to an inverse Radon transform operation includes:
determining, by back-projecting the second derivative of Radon transform data on the meridian plane of 0-π or π-2π according to the inverse Radon transform operation, the reconstructed image of the subject.

14. A method, the method being implemented on a computing device having at least one storage device and at least one processor, the method comprising:
obtaining projection data of a subject corresponding to each of at least one projection angle;
for the projection data corresponding to each of the at least one projection angle, determining a weight corresponding to each of detection angles within a projection range;
determining, based on the weight and the projection data, a first derivative of Radon transform data; and
determining, based on the first derivative of the Radon transform data, a reconstructed image of the subject.

15. A computer-readable storage medium, the storage medium stores computer instructions, when the computer instructions are executed by the processor to implement the methods including:
obtaining projection data of a subject corresponding to each of at least one projection angle;
for the projection data corresponding to each of the at least one projection angle, determining a weight corresponding to each of detection angles within a projection range;
determining, based on the weight and the projection data, a first derivative of Radon transform data; and
determining, based on the first derivative of the Radon transform data, a reconstructed image of the subject.
